SEQUENCE LISTING (23 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 218 aa, Artificial Sequence
  Ab6 murine humanized light chain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Artificial Sequence
  Ab6 murine humanized heavy chain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 111 aa, Artificial Sequence
  Ab6 murine humanized light chain immunoglobulin variable domain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 5 - protein, 119 aa, Artificial Sequence
  Ab6 murine humanized heavy chain immunoglobulin variable domain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 6 - protein, 15 aa, Artificial Sequence
  Description of Artificial Sequence Synthetic peptide
  Ab6 murine CDR-L1
KASQSLDYEGDSDMN

SEQ ID NO: 7 - protein, 7 aa, Artificial Sequence
  Description of Artificial Sequence Synthetic peptide
  Ab6 murine CDR-L2
GASNLES

SEQ ID NO: 8 - protein, 9 aa, Artificial Sequence
  Description of Artificial Sequence Synthetic peptide
  Ab6 murine CDR-L3
QQSTEDPRT

SEQ ID NO: 9 - protein, 5 aa, Artificial Sequence
  Description of Artificial Sequence Synthetic peptide
  Ab6 murine CDR-H1
DYNVD

SEQ ID NO: 10 - protein, 17 aa, Artificial Sequence
  Description of Artificial Sequence Synthetic peptide
  Ab6 murine humanized CDR-H2
DINPNDGGTIYAQKFQE

SEQ ID NO: 11 - protein, 10 aa, Artificial Sequence
  Description of Artificial Sequence Synthetic peptide
  Ab6 murine CDR-H3
NYRWFGAMDH

SEQ ID NO: 12 - DNA, 25 bp, Artificial Sequence
  Description of Artificial Sequence Synthetic oligonucleotide
  Oligonucleotide
gaggaaaatttgtttcatacagaag

SEQ ID NO: 13 - DNA, 12 bp, Artificial Sequence
  Description of Artificial Sequence Synthetic oligonucleotide
  Oligonucleotide
agggatttcacc

SEQ ID NO: 14 - DNA, 17 bp, Artificial Sequence
  Description of Artificial Sequence Synthetic oligonucleotide
  Oligonucleotide
tatgtgtaatatgtaaa

SEQ ID NO: 15 - DNA, 15 bp, Artificial Sequence
  Description of Artificial Sequence Synthetic oligonucleotide
  Oligonucleotide
attgtatgaattaaa

SEQ ID NO: 16 - protein, 529 aa, Artificial Sequence
  Description of Artificial Sequence Synthetic polypeptide
  human LAG3 protein with VEGF leader
MNFLLSWVHWSLALLLYLHHAKWSQALQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 17 - DNA, 637 bp, Artificial Sequence
  Description of Artificial Sequence Synthetic polynucleotide
  Mouse IL-2 promotor
ggtaccgtacctcaagctcaacaagcattttaggtgtccttagcttactatttctctggctaactgtatgaagccatctatcaccctgtgtgcaattagctcattgtgtagataagaaggtaaaaccatcttgaaacaggaaaccaatatccttcctgtctaatcaacaaatctaaaagatttattcttttcatctatctcctcttgcgtttgtccaccacaacaggctgcttacaggttcaggatggttttgacaaagagaacattttcatgagttacttttgtgtctccaccccaaagaggaaaatttgtttcatacagaaggcgttcattgtatgaattaaaactgccacctaagtgtgggctaacccgaccaagagggatttcacctaaatccattcagtcagtgtatgggggtttaaagaaattccagagagtcatcagaagaggaaaaacaaaggtaatgctttctgccacacaggtagactctttgaaaatatgtgtaatatgtaaaacatcgtgacacccccatattatttttccagcattaacagtataaattgcctcccatgctgaagagctgcctatcacccttgctaatcactcctcacagtgacctcaagtcctgcagaagctt

SEQ ID NO: 18 - DNA, 625 bp, Artificial Sequence
  Description of Artificial Sequence Synthetic polynucleotide
  mouse IL-2 promotor
gtacctcaagctcaacaagcattttaggtgtccttagcttactatttctctggctaactgtatgaagccatctatcaccctgtgtgcaattagctcattgtgtagataagaaggtaaaaccatcttgaaacaggaaaccaatatccttcctgtctaatcaacaaatctaaaagatttattcttttcatctatctcctcttgcgtttgtccaccacaacaggctgcttacaggttcaggatggttttgacaaagagaacattttcatgagttacttttgtgtctccaccccaaagaggaaaatttgtttcatacagaaggcgttcattgtatgaattaaaactgccacctaagtgtgggctaacccgaccaagagggatttcacctaaatccattcagtcagtgtatgggggtttaaagaaattccagagagtcatcagaagaggaaaaacaaaggtaatgctttctgccacacaggtagactctttgaaaatatgtgtaatatgtaaaacatcgtgacacccccatattatttttccagcattaacagtataaattgcctcccatgctgaagagctgcctatcacccttgctaatcactcctcacagtgacctcaagtcctgcag

SEQ ID NO: 19 - DNA, 624 bp, Artificial Sequence
  Description of Artificial Sequence Synthetic polynucleotide
  Mouse IL-2 promotor
tacctcaagctcaacaagcattttaggtgtccttagcttactatttctctggctaactgtatgaagccatctatcaccctgtgtgcaattagctcattgtgtagataagaaggtaaaaccatcttgaaacaggaaaccaatatccttcctgtctaatcaacaaatctaaaagatttattcttttcatctatctcctcttgcgtttgtccaccacaacaggctgcttacaggttcaggatggttttgacaaagagaacattttcatgagttacttttgtgtctccaccccaaagaggaaaatttgtttcatacagaaggcgttcattgtatgaattaaaactgccacctaagtgtgggctaacccgaccaagagggatttcacctaaatccattcagtcagtgtatgggggtttaaagaaattccagagagtcatcagaagaggaaaaacaaaggtaatgctttctgccacacaggtagactctttgaaaatatgtgtaatatgtaaaacatcgtgacacccccatattatttttccagcattaacagtataaattgcctcccatgctgaagagctgcctatcacccttgctaatcactcctcacagtgacctcaagtcctgcag

SEQ ID NO: 20 - protein, 16 aa, Artificial Sequence
  Description of Artificial Sequence Synthetic peptide
  Synthetic HEL peptide
DGSTDYGILQINSRWW

SEQ ID NO: 21 - protein, 16 aa, Artificial Sequence
  Description of Artificial Sequence Synthetic peptide
  Synthetic HEL peptide
NTDGSTDYGILQINSR

SEQ ID NO: 22 - protein, 15 aa, Artificial Sequence
  Description of Artificial Sequence Synthetic peptide
  Synthetic HEL peptide
DGSTDYGILQINSRW

SEQ ID NO: 23 - protein, 26 aa, Artificial Sequence
  Description of Artificial Sequence Synthetic peptide
  synthetic VEGF leader peptide
MNFLLSWVHWSLALLLYLHHAKWSQA

(19) United States
(12) Patent Application Publication
(10) Pub. No.: US 2023/0333090 A1
(43) Pub. Date: Oct. 19, 2023

(54) LAG3 ANTAGONIST CELL BASED POTENCY ASSAY

(71) Applicant: Merck Sharp & Dohme LLC, Rahway, NJ (US)

(72) Inventors: Kevin B. Gurney, Vineyard, UT (US); Yue Zhang, Warren, NJ (US); Linda Liang, Mountain View, CA (US); Cuiwei Ai, Sunnyvale, CA (US)

(73) Assignee: Merck Sharp & Dohme LLC, Rahway, NJ (US)

(21) Appl. No.: 17/786,039

(22) PCT Filed: Dec. 16, 2020
(86) PCT No.: PCT/US2020/065294

§ 371 Date: Jun. 16, 2022

Related U.S. Application Data
(60) Provisional application No. 62/951,484, filed on Dec. 20, 2019.

(51) Int. Cl. G01N 33/50; C12N 5/0783

(52) U.S. Cl. CPC G01N 33/505; C12N 5/0636; G01N 33/5023; G01N 2333/55; G01N 2333/70503

(57) ABSTRACT

The present disclosure describes a murine T cell 3A9 comprising a polynucleotide sequence encoding human LAG3, and a murine IL-2 promotor operably associated with a reporter gene. The disclosure also describes a cell-based potency assay for measuring LAG3 antagonist activity using a co-cultured system with the murine T cells and LK35.2 murine B cells, and hen egg lysozyme peptide.

### FIELD OF THE INVENTION

The present disclosure describes a murine T cell 3A9 comprising a polynucleotide sequence encoding human LAG3, and a murine IL-2 promotor operably associated with a reporter gene. The disclosure also describes a cell-based potency assay for measuring LAG3 antagonist activity using a co-cultured system with the murine T cells and LK35.2 murine B cells, and hen egg lysozyme peptide.

### REFERENCE TO SEQUENCE LISTING SUBMITTED ELECTRONICALLY

The sequence listing of the present application is submitted electronically via EFS-Web as an ASCII formatted sequence listing with a file name 24944-US-PCT SL.txt, creation date of Jun. 26, 2023, and a size of 25,012 bytes. This sequence listing submitted via EFS-Web is part of the specification and is herein incorporated by reference in its entirety.

### BACKGROUND OF THE INVENTION

LAG-3 (Lymphocyte Activation Gene-3) is a cell surface molecule expressed on activated T cells, B cells, NK cells, and plasmacytoid dendritic cells. LAG-3 is structurally similar to CD4, and binds to MHC class II molecules as an inhibitory receptor. LAG-3 was shown to negatively regulate T-cell activation and proliferation, as well as to be co-expressed on tumor-infiltrating lymphocytes with other inhibitory receptors. Expression of LAG3 is indicative of a highly exhausted T-cell phenotype. See Goldberg MV1, Drake C G. Curr. Top. Microbiol. Immunol. 2011; 344:269-78.

Several anti-LAG3 antibodies are in clinical development, and there is a need for LAG3 cell-based assays to confirm potency of clinical batches of anti-LAG3 antibodies. A previously developed first generation co-culture system requires the use of Staphylococcal Enterotoxins (SED or SEE). SED and SEE are highly regulated substances and are classified as select agents in the United States. The incorporation of Staph Enterotoxins in a lot release assay could severely hamper the LAG-3 product release in the GMP space due to supply issues or regulatory restrictions for its use.

### SUMMARY OF THE INVENTION

The present invention provides a cell-based potency assay for measuring LAG3 antagonist activity using a co-cultured system with murine 3A9 T cells and LK35.2 murine B cells, and hen egg lysozyme peptide. The murine T cell 3A9 (ATCC deposit CRL-3293) comprises a polynucleotide sequence encoding human LAG3, a murine IL-2 promotor operably associated with a reporter gene, wherein the mature human LAG3 is expressed on the cell surface. A murine IL-2 promotor sequence consisting of nucleotides −580 to +45 (SEQ ID NO: 18) or -579 to +45 (SEQ ID NO: 19) can be used. The assay is robust, highly quantitative and accurate.

### BRIEF DESCRIPTION OF THE DRAWINGS

FIG. 1 Schematic map of parental promoterless vector pGL4.15. from Promega

FIG. 2 Initial pooled cells study comparing hIL-2 or mIL-2 promoters. The mIL2-Luc group 1 and 2 are two treatments using the same cells 3A9-hLAG3-mIL2-Luc2P. Parental is the 3A9-hLAG3 cell line only without adding mIL2-Luc2P. huIL2-Luc group 2 is treatment with 3A9-hLAG3-hIL2-Luc2P. RLU is relative luminescence unit.

FIG. 3 LAG-3 luciferase assay using 3A9-hLAG3-mIL2-Luc2P stable cell line; Relative Potency (%) of anti-LAG-3 monoclonal antibody Ab6 drug substance batches against Ab6 reference batch as measured by cell-based assay. Error bars reflect upper and lower 95% confidence levels.

FIG. 4 Interactions of HEL peptide and MHC class II molecule I-A^{k}. Figure discloses SEQ ID NO: 22.

FIG. 5 Comparison of different potency results using various HEL peptide sequences.

FIG. 6A-B A: ONE-Glo luciferase assay substrate reaction principle; B: Principle of luciferase reporter transcriptional and translational steps in cell-based assay.

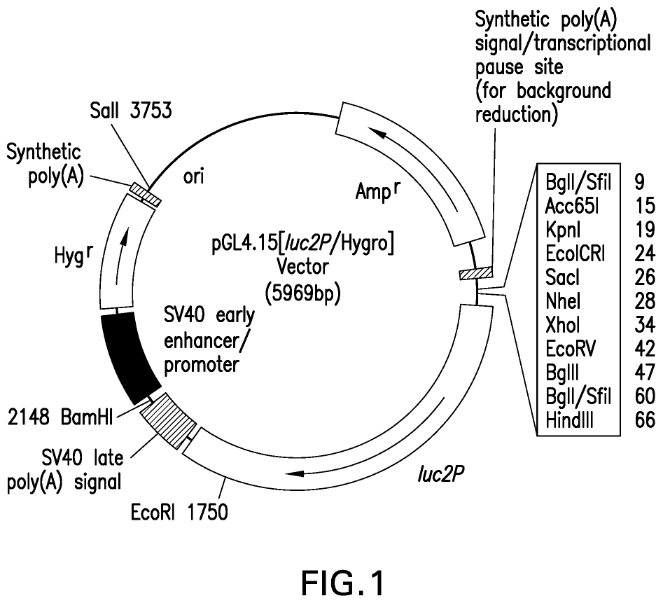

### DETAILED DESCRIPTION

Abbreviations. Throughout the detailed description and examples of the invention the following abbreviations will be used:

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, an “Ab6 variant” means a monoclonal antibody which comprises heavy chain and light chain sequences that are substantially identical to those in antibody Ab6 (as described below and in WO2016028672, incorporated by reference in its entirety), except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region of the immunoglobulin chain(s), and optionally has a deletion of the C-terminal lysine residue of the heavy chain. In other words, Ab6 and a Ab6 variant comprise identical CDR sequences, but differ from each other due to having a conservative amino acid substitution at no more than three or six other amino acid positions in the full length light and heavy chain sequences, respectively. An Ab6 variant is substantially the same as Ab6 with respect to the following properties: binding affinity to human LAG3 and ability to block the binding of human LAG3 to human MHC Class II.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a LAG3 antagonist that consists essentially of a recited amino acid sequence may also include one or more amino acids, including substitutions of one or more amino acid residues, which do not materially affect the properties of the binding compound.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“LAG3 antagonist” means any chemical compound or biological molecule that blocks binding of LAG3 expressed on an immune cell (T cell, Tregs, or NK cell etc.) to MHC Class II molecules. Human LAG3 comprises the amino acid sequence:

(SEQ ID NO: 1)
MWEAQFLGLL FLQPLWVAPV KPLQPGAEVP VVWAQEGAPA
QLPCSPTIPL QDLSLLRRAG VTWQHQPDSG PPAAAPGHPL
APGPHPAAPS SWGPRPRRYT VLSVGPGGLR SGRLPLQPRV
QLDERGRORG DFSLWLRPAR RADAGEYRAA VHLRDRALSC
RLRLRLGQAS MTASPPGSLR ASDWVILNCS FSRPDRPASV
HWFRNRGQGR VPVRESPHHH LAESFLFLPQ VSPMDSGPWG
CILTYRDGFN VSIMYNLTVL GLEPPTPLTV YAGAGSRVGL
PCRLPAGVGT RSFLTAKWTP PGGGPDLLVT GDNGDFTLRL
EDVSQAQAGT YTCHIHLQEQ QLNATVTLAI ITVTPKSFGS
PGSLGKLLCE VTPVSGQERF VWSSLDTPSQ RSFSGPWLEA
QEAQLLSQPW QCQLYQGERL LGAAVYFTEL SSPGAQRSGR
APGALPAGHL LLFLILGVLS LLLLVTGAFG FHLWRRQWRP
RRFSALEQGI HPPQAQSKIE ELEQEPEPEP EPEPEPEPEP EPEQL;
see also Uniprot accession no. P18627.
Residues 1-22 are the native leader sequence.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A “coding sequence” or a sequence “encoding” an expression product, such as a RNA, polypeptide, protein, or enzyme, is a nucleotide sequence that, when expressed, results in production of the product.

The term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a reporter gene.

A coding sequence is “under the control of”, “functionally associated with” or “operably associated with” transcriptional and translational control sequences in a cell when the sequences direct RNA polymerase mediated transcription of the coding sequence into RNA, preferably mRNA, which then may be RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “upstream” means that the gene is to the 5′ end of the other gene.

The term “transformation” means the introduction of a nucleic acid into a cell. The introduced gene or sequence may be called a “clone”. A host cell that receives the introduced DNA or RNA has been “transformed” and is a “transformant” or a “clone.” The DNA or RNA introduced to a host cell can come from any source, including cells of the same genus or species as the host cell, or from cells of a different genus or species.

“Reporter Gene” refers to a gene that is attached to a regulatory sequence of another gene of interest and used to indicate whether the gene of interest has been expressed in the cell. Examples of reporter genes include but are not limited to β-lactamase, which catalyzes hydrolysis of a cephalosporin monitored by a change in fluorescence emission of a substrate (Zlokarnik et al., Science, 279 (1998), pp. 84-88); firefly luciferase, which oxidizes luciferin, emitting photons, and Renilla luciferase, which catalyzes oxidation of coelentrazine, leading to bioluminescence (Inouye and Shimomura, Biochem. Biophys. Res. Commun., 233 (1997), pp. 349-353); and green fluorescent protein (GFP), which fluoresces, due to energy transfer, on irradiation (Kunert et al., J. Microbiol. Methods, 41 (2000), pp. 185-194).

Various firefly luciferase genes are commercially available for use in the invention. Promoterless firefly luciferase vectors from Promega are designed primarily to accept a putative promoter element for investigation of important regions controlling gene transcription. The promoterless vectors are available with three varieties of engineered firefly luciferase genes: luc2, luc2P or luc2CP. The luc2 gene is engineered to remove most cryptic transcription factor binding sites and improve mammalian expression through codon optimization. The luc2P and luc2CP and RapidResponse™ genes are luc2 genes appended with degradation sequences to influence the cellular half-life of the luc2 gene. The RapidResponse™ genes respond more rapidly to stimuli but at the expense of signal intensity. The luc2P (1-hour half-life) gene responds more rapidly than luc2 (3-hour half-life) with moderate signal intensity, and the luc2CP (0.4-hour half-life) responds more quickly with the lowest signal intensity. The promoterless vectors are available with or without selectable markers (hygromycin, neomycin or puromycin).

“Read-out signal” refers to a signal produced from the reporter gene protein expression. The signal can be emitted by the protein or reaction of the protein with a substrate. In one embodiment, the signal is fluorescence or luminescence.

“Stably transfected” refers to the foreign gene being part of the host genome and is therefore replicated. This is typically initiated by transiently transfecting a cell with the foreign gene but through a process of careful selection and amplification, and stable clones are generated. One method to select for stable clones is to use selectable markers expressed on the plasmid DNA to enable the selection of cells that have successfully integrated the gene into their genome. A common method used is to design the plasmid DNA to also contain a gene that expresses antibiotic resistance. Continued antibiotic treatment of the cells for long-term results in the expansion of only the stably-transfected cells. Descendants of these stably-transfected cells, also express the foreign gene, resulting in a stably transfected cell line.

The term “vector” includes a vehicle (e.g., a plasmid) by which a DNA or RNA sequence can be introduced into a host cell, so as to transform the host and, optionally, promote expression and/or replication of the introduced sequence.

“Assay media” refers to a solution comprising a nutrients for cells such as glucose, vitamins, amino acids, or a combination thereof and serum, and optionally antibiotics and a buffer.

The 3A9-hLAG3-mIL2-Luc2P stable cell line was generated as a potency reporter cell line to test LAG-3 antagonists to evaluate LAG-3 function. The cell line was engineered with (1) human LAG-3 stably expressed on the cell surface and (2) a murine interleukin-2 (IL-2) promoter that is upstream of a firefly luciferase gene stably designed intracellularly. See Goldberg MV1, Drake C G. Curr. Top. Microbiol. Immunol. 2011; 344:269-78. The cell line is used in an anti-LAG3 functional cell-based assay that utilizes a co-culture of the 3A9 T-cells, which are engineered to stably express the human LAG3 receptor (CD223) and contain the IL-2 promoter linked to the luciferase reporter gene, with LK35.2 B-cells in the presence of a HEL (hen egg lysozyme) peptide. The peptide binds to the MHC II (class II major histocompatibility complex) molecule, I-Ak, on LK35.2 B cells and induces activation of the 3A9 T-cells. LAG-3 binds to the MHC II complex as an inhibitory receptor, negatively regulating T-cell activation. Introduction of a LAG3 antagonist antibody blocks this binding and restores T-cell activation, which is measured by production of IL-2 promoter-driven luciferase enzyme. The T-cell activation is directly proportional to the anti-LAG-3 activity of the LAG3 antagonist bound to LAG-3 receptor and is determined by measuring luminescence produced after incubation with a luciferase substrate.

The present invention provides a murine T cell 3A9 (ATCC deposit CRL-3293) comprising a polynucleotide sequence encoding human LAG3, a polynucleotide sequence comprising a murine IL-2 promotor operably associated with a reporter gene, wherein the mature human LAG3 is expressed on the cell surface. In one embodiment, the polynucleotide sequences are stably transfected. In one embodiment, the murine IL-2 promotor sequence consists of one or more binding sites for NFAT-1 (SEQ ID NO:12), NFkB (SEQ ID NO: 13), NF-IL2A (SEQ ID NO: 14 or SEQ ID NO: 15) and AP-1 (TCAGTCA). In one embodiment, there can be two, three, four or five repeats of one or more of the binding sites. In another embodiment, the murine IL-2 promotor sequence consists of nucleotides −580 to +45 (SEQ ID NO: 18) or −579 to +45 (SEQ ID NO: 19). In another embodiment, the reporter gene is a firefly luciferase gene or green fluorescence protein gene. In another embodiment, the human LAG3 sequence is SEQ ID NO: 16.

The invention also provides a method for measuring the activity of a LAG3 antagonist comprising the steps of:
a) Combining suspensions of the murine T cells of the invention and LK35.2 B cells with a Hen Egg Lysozyme (HEL) peptide of SEQ ID NO: 20, 21 or 22, and a LAG3 antagonist in an assay media; and
b) Detecting the reporter gene expression through a read-out signal.

In one embodiment, equal volumes of the murine T cells at a concentration of 3×10⁶ cells/ml and LK35.2 cells at a concentration of 0.75×10⁶ cells/ml are combined. The suspensions of cells can be prepared by culturing the cells in growth medium, harvesting the cells, and diluting to the desired concentration. In one embodiment, the HEL peptide is SEQ ID NO: 20.

In another embodiment, the LAG3 antagonist is both a reference standard and a test sample, and the method further comprises the step of determining the relative potency of the test sample to the reference standard. In another embodiment, the reporter gene is firefly luciferase gene, and 5′-fluoroluciferin is added and the luminescence is detected.

### LAG3 Antagonist

LAG3 antagonists useful in the method of the present invention include a monoclonal antibody (mAb), or antigen binding fragment thereof, which specifically binds to LAG3. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

In one embodiment, the anti-LAG3 antibody is Ab6 or an Ab6 variant. Ab6 has the following antibody components:

a light chain immunoglobulin comprising the amino
acid sequence:
(SEQ ID NO: 2)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;
a heavy chain immunoglobulin comprising the amino
acid sequence:
(SEQ ID NO: 3)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;
a light chain immunoglobulin variable domain
comprising the amino acid sequence:
(SEQ ID NO: 4 (CDRs underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
a heavy chain immunoglobulin variable domain
comprising the amino acid sequence:
(SEQ ID NO: 5 (CDRs underscored))
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSS;
and the following CDRs:
CDR-L1:
(SEQ ID NO: 6)
KASQSLDYEGDSDMN;
CDR-L2:
(SEQ ID NO: 7)
GASNLES;
CDR-L3:
(SEQ ID NO: 8)
QQSTEDPRT;
CDR-H1:
(SEQ ID NO: 9)
DYNVD;
CDR-H2:
(SEQ ID NO: 10)
DINPNDGGTIYAQKFQE;
and
CDR-H3:
(SEQ ID NO: 11)
NYRWEGAMDH

In some preferred embodiments of the method of the present invention, the LAG3 antagonist is a monoclonal antibody, or antigen binding fragment thereof, which comprises: (a) light chain CDRs SEQ ID NOs: 6, 7 and 8, and (b) heavy chain CDRs SEQ ID NOs: 9, 10 and 11.

In other preferred embodiments of the method of the present invention, the LAG3 antagonist is a monoclonal antibody, or antigen binding fragment thereof, which specifically binds to human LAG3 and comprises (a) a heavy chain variable region comprising SEQ ID NO:5 or a variant thereof, and (b) a light chain variable region comprising SEQ ID NO:4 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the method of the present invention, the LAG3 antagonist is a monoclonal antibody which specifically binds to human LAG3 and comprises (a) a heavy chain comprising SEQ ID NO: 3 and (b) a light chain comprising SEQ ID NO:2. In another preferred embodiment of the method of the present invention, the LAG3 antagonist is a monoclonal antibody which specifically binds to human LAG3 and comprises (a) a heavy chain variable region comprising SEQ ID NO: 5 and (b) a light chain variable region comprising SEQ ID NO:4.

Other Examples of mAbs that bind to human LAG3, and useful in the method of the present invention, are relatlimab, IMP731, IMP701, anti-LAG3 antibodies disclosed in US2017101472. Other LAG3 antagonists useful in the method of the present invention include an immunoadhesin that specifically binds to human LAG3, e.g., a fusion protein containing the extracellular LAG3 fused to a constant region such as an Fc region of an immunoglobulin molecule.

In one embodiment, the anti-LAG3 antibody or antigen-binding fragment comprises a heavy chain constant region, e.g. a human constant region, such as γ1, γ2, γ3, or γ4 human heavy chain constant region or a variant thereof. In another embodiment, the anti-LAG3 antibody or antigen-binding fragment comprises a light chain constant region, e.g. a human light chain constant region, such as lambda or kappa human light chain region or variant thereof. By way of example, and not limitation, the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols.1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

### EXAMPLES

3A9-hLAG3-mIL2-Luc2P cell line was generated based upon stable pooled cell line 3A9-hLAG3. Retroviral vector plasmid containing human LAG3 (pMXpuro-hLAG3) was codon optimized and engineered. The plasmid was transduced into murine 3A9 T cells and a stable pool was generated with Puromycin selection. 3A9-hLAG3 single clone cell lines were generated by a functional assay screening of 90 single clones. A mouse IL-2 promoter driven luciferase was engineered and added into 3A9-hLAG3 3F8 clone to make a stable pool. Single clonal cell line 3A9-hLAG3-mIL2-Luc2P clone 7D1 was selected as the final cell line for LAG-3 functional assay.

The murine cell line LK35.2 (ATCC HB-98, Kappler J, et al. Proc. Natl. Acad. Sci. USA 79: 3604-3607, 1982), was used as a co-cultured antigen presenting cell (APC) line to provide MHC II complex to the 3A9 T cell. LK35.2 was originated by fusing A20.2J lymphoma cells (I-A^{d}, I-E^{d}) with T cell depleted spleen cells from B10.BR mice (I-A^{k}, I-E^{k}). LK35.2 cells bear surface I-A^{d,k} and I-E^{d,k} molecules and can present antigen to appropriate I region restricted T cell hybridomas.

### Example 1: Plasmid Design and Generation

mIL-2 Promoter Design

The mouse IL-2 promoter sequence (retrieved from GenBank Accession #X52618) was designed based on the homology between mouse and human IL-2 gene sequence. See Novak, T J et., al, 1990, Nucleic Acids Research, vol. 18, No. 15 4523. Mouse IL-2 gene +1 to −580 bp has ˜86% sequence homology comparing to previously reported human sequence. Proximal sites which were shown to be important in regulation of the human IL-2 gene are well conserved in sequence and location (−279 to −1). This includes binding sites for NFAT-1 (−287 to −263, GAGGAAAATTTGTTTCATACAGAAG SEQ ID NO:12), NFkB (−208 to −197, AGGGATTTCACC SEQ ID NO: 13), NF-IL2A (−89 to −73, TATGTGTAATATGTAAA SEQ ID NO: 14; −256 to −241, ATTGTATGAATTAAA SEQ ID NO: 15) and AP-1 (−187 to −181, TCAGTCA), etc. The negative regulatory region lying more upstream (between −578 and −1219, poly d(CA) sequence) was intentionally removed to prevent the potential feedback loop regulation. Sequence (−580 to +45) was synthesized and Kpnl and HindIll restriction sites were added as flanking sequences for cloning. The −580 to +45 sequence with restrictions sites underlined is shown below:

(SEQ ID NO: 17)
ggtaccGTACCTCAAGCTCAACAAGCATTTTAGGTGTCCTTAGCTTACTA
TTTCTCTGGCTAACTGTATGAAGCCATCTATCACCCTGTGTGCAATTAGC
TCATTGTGTAGATAAGAAGGTAAAACCATCTTGAAACAGGAAACCAATAT
CCTTCCTGTCTAATCAACAAATCTAAAAGATTTATTCTTTTCATCTATCT
CCTCTTGCGTTTGTCCACCACAACAGGCTGCTTACAGGTTCAGGATGGTT
TTGACAAAGAGAACATTTTCATGAGTTACTTTTGTGTCTCCACCCCAAAG
AGGAAAATTTGTTTCATACAGAAGGCGTTCATTGTATGAATTAAAACTGC
CACCTAAGTGTGGGCTAACCCGACCAAGAGGGATTTCACCTAAATCCATT
CAGTCAGTGTATGGGGGTTTAAAGAAATTCCAGAGAGTCATCAGAAGAGG
AAAAACAAAGGTAATGCTTTCTGCCACACAGGTAGACTCTTTGAAAATAT
GTGTAATATGTAAAACATCGTGACACCCCCATATTATTTTTCCAGCATTA
ACAGTATAAATTGCCTCCCATGCTGAAGAGCTGCCTATCACCCTTGCTAA
TCACTCCTCACAGTGACCTCAAGTCCTGCAGaagctt

The −580 to +45 sequence without restriction sites, is shown below:

(SEQ ID NO: 18)
GTACCTCAAGCTCAACAAGCATTTTAGGTGTCCTTAGCTTACTATTTCTC
TGGCTAACTGTATGAAGCCATCTATCACCCTGTGTGCAATTAGCTCATTG
TGTAGATAAGAAGGTAAAACCATCTTGAAACAGGAAACCAATATCCTTCC
TGTCTAATCAACAAATCTAAAAGATTTATTCTTTTCATCTATCTCCTCTT
GCGTTTGTCCACCACAACAGGCTGCTTACAGGTTCAGGATGGTTTTGACA
AAGAGAACATTTTCATGAGTTACTTTTGTGTCTCCACCCCAAAGAGGAAA
ATTTGTTTCATACAGAAGGCGTTCATTGTATGAATTAAAACTGCCACCTA
AGTGTGGGCTAACCCGACCAAGAGGGATTTCACCTAAATCCATTCAGTCA
GTGTATGGGGGTTTAAAGAAATTCCAGAGAGTCATCAGAAGAGGAAAAAC
AAAGGTAATGCTTTCTGCCACACAGGTAGACTCTTTGAAAATATGTGTAA
TATGTAAAACATCGTGACACCCCCATATTATTTTTCCAGCATTAACAGTA
TAAATTGCCTCCCATGCTGAAGAGCTGCCTATCACCCTTGCTAATCACTC
CTCACAGTGACCTCAAGTCCTGCAG

The −579 to +45 sequence is shown below:

(SEQ ID NO: 19)
TACCTCAAGCTCAACAAGCATTTTAGGTGTCCTTAGCTTACTATTTCTCT
GGCTAACTGTATGAAGCCATCTATCACCCTGTGTGCAATTAGCTCATTGT
GTAGATAAGAAGGTAAAACCATCTTGAAACAGGAAACCAATATCCTTCCT
GTCTAATCAACAAATCTAAAAGATTTATTCTTTTCATCTATCTCCTCTTG
CGTTTGTCCACCACAACAGGCTGCTTACAGGTTCAGGATGGTTTTGACAA
AGAGAACATTTTCATGAGTTACTTTTGTGTCTCCACCCCAAAGAGGAAAA
TTTGTTTCATACAGAAGGCGTTCATTGTATGAATTAAAACTGCCACCTAA
GTGTGGGCTAACCCGACCAAGAGGGATTTCACCTAAATCCATTCAGTCAG
TGTATGGGGGTTTAAAGAAATTCCAGAGAGTCATCAGAAGAGGAAAAACA
AAGGTAATGCTTTCTGCCACACAGGTAGACTCTTTGAAAATATGTGTAAT
ATGTAAAACATCGTGACACCCCCATATTATTTTTCCAGCATTAACAGTAT
AAATTGCCTCCCATGCTGAAGAGCTGCCTATCACCCTTGCTAATCACTCC
TCACAGTGACCTCAAGTCCTGCAG

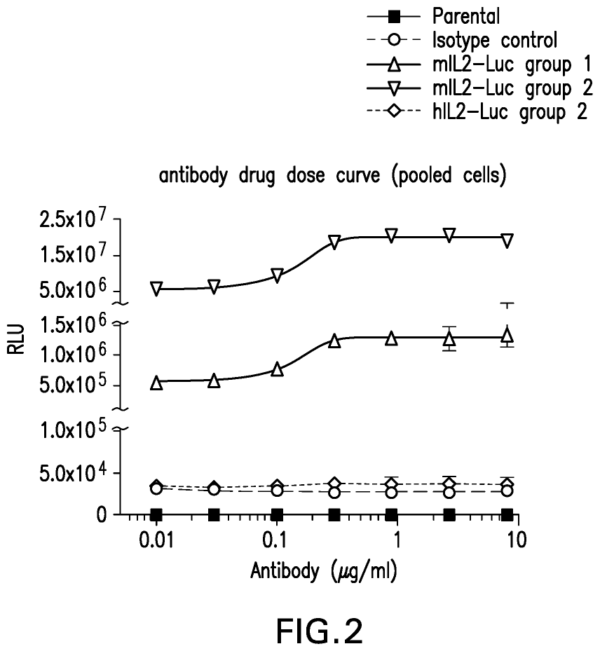

It is worth noting that mouse IL-2 promoter region is specific to drive luciferase expression in the 3A9 cell line system. A separate study using a human IL-2 promoter (pGL4[luc2P/IL-2/Hygro] Vector from Promega) yielded negative results (FIG. 2). The two vectors have identical backbones and other components except human or mouse IL-2 promoters.

### Luciferase Vector Selection:

pGL4.15 (luc2/Hygro) vector is a new generation promoterless vector designed by removing potential cryptic promoters. The vector expresses firefly luciferase and the sequence is codon optimized. A PEST degradation sequence is added to the C-terminus of luciferase to shorten luciferase half-life, which can improve assay window. See Promoterless Firefly Luciferase Vectors with Hygromycin Selection, Promega Cat #E6701.

### Example 2: Cell Line Generation

### 3A9 Cell Line Information

3A9 cell line is a mouse T cell line from ATCC (Cat #CRL-3293). The cell line was generated from CBA/J mice, and T-lymphocytes were fused with BW5147 T cell lymphoma cells. See Allen PM, Journal of Immunology Vol 132, No. 3, 1984. 3A9 cell line expresses CD3/TCR and CD4.

### 3A9-hLAG3 Stable Pool Cell Line Generation

Retroviral vector plasmid containing human LAG3 (pMXpuro-hLAG3) was codon optimized and engineered. A VEGF leader sequence (MNFLLSWVHWSLALLLYLHHAKWSQA SEQ ID NO: 23) was used instead of the native human LAG3 leader sequence for better expression. A stable pool cell line 3A9-hLAG3 was 15 generated by transient transfection of 3A9 cells with the hLAG3 plasmid; the cell pool underwent puromycin selection to form a stable pool. The human LAG3 protein with VEGF leader sequence is:

(SEQ ID NO: 16)
MNFLLSWVHWSLALLLYLHHAKWSQALQPGAEVPVVWAQEGAPAQLPCSP
TIPLQDLSLLRRAGVTWQHQPDSGPPAAAPGHPLAPGPHPAAPSSWGPRP
RRYTVLSVGPGGLRSGRLPLQPRVQLDERGRQRGDFSLWLRPARRADAGE
YRAAVHLRDRALSCRLRLRLGQASMTASPPGSLRASDWVILNCSFSRPDR
PASVHWFRNRGQGRVPVRESPHHHLAESFLFLPQVSPMDSGPWGCILTYR
DGFNVSIMYNLTVLGLEPPTPLTVYAGAGSRVGLPCRLPAGVGTRSFLTA
KWTPPGGGPDLLVTGDNGDFTLRLEDVSQAQAGTYTCHIHLQEQQLNATV
TLAIITVTPKSFGSPGSLGKLLCEVTPVSGQERFVWSSLDTPSQRSFSGP
WLEAQEAQLLSQPWQCQLYQGERLLGAAVYFTELSSPGAQRSGRAPGALP
AGHLLLFLILGVLSLLLLVTGAFGFHLWRRQWRPRRFSALEQGIHPPQAQ
SKIEELEQEPEPEPEPEPEPEPEPEPEQL

### 3A9-hLAG3 Clonal Cell Line Generation

Early passage 3A9-hLAG3 pool cells underwent limiting dilution to generate single clones. 90 single clones were chosen, screened by a cell-based functional assay, and ranked according to the performance criteria. The functional assay was an AlphaLISA based homogenous assay measuring IL-2 secretion. The final clone selected was 3A9-hLAG3 3F8.

3A9-hLAG3 mIL2-Luc2P Stable Pool Cell Line Generation

Clones 3A9-hLAG3 3F8 were electroporated by Lonza 4D-Nucleofector, using Nucleofector kits for Jurkat (Cat #V4XC-1024), and CL-120 program. Plasmid mIL2-Luc2p at 1 μg and 2 μg were electroporated into clone 3F8. Plasmid pMaxGFP was used as a control. Electroporated cells were recovered in media without antibiotics selection for 48 hrs, and subsequently 1000 μg/ml Hygromycin was added for selection. Cell viability was examined every 3-5 days, until stable pool was formed.

3A9-hLAG3 mIL2-Luc2P Clonal Cell Line Generation

The stable pool underwent limiting dilution to generate single clones. 140 single clones were grown up, screened by a cell-based functional assay, and ranked according to the performance criteria. The functional assay was a reporter based luciferase assay measuring luciferase activity. The final clone selected was 3A9-hLAG3-mIL2-Luc2P 7D1.

3A9-hLAG3 mIL2-Luc2P Clone 7D1 Characterization

Clone 3A9-hLAG3-mIL2-Luc2P 7D1 was banked at early passage P4 for initial repository. Clone 7D1 was further characterized by continuously monitoring doubling time and assay function over 3 months period. The 7D1 cell line was considered stable by comparing results within the 3 months timeframe.

### Growth Media

Growth media should be prepared as 1 μg/mL Puromycin, 750 μg/mL Hygromycin, 100 U/mL Penicillin-Streptomycin, 10 mM HEPES, 10% Heat Inactivated FBS in RPMI 1640 with GlutaMAX supplement.

TABLE 2
Example preparation of growth media
for 3A9-hLAG3-mIL2-Luc2P 7D1

| | | | Volume | Final | |
|---|---|---|---|---|---|
| Reagent | Cat. #. | Vendor | [mL] | concentration | |
| RPMI 1640, | 61870-036 | Life | 432.5 | 86.5% | |
| GlutaMAX | | Technologies | | | |
| supplement | | | | | |
| FBS, heat | 16140-071 | Life | 50 | 10% | |
| inactivated | | Technologies | | | |
| Pennicillin- | 15140-122 | Life | 5 | 100 | U/mL |
| Streptomycin | | Technologies | | | |
| HEPES | 15630-080 | Life | 5 | 10 | mM |
| | | Technologies | | | |
| Hygromycin | 10687010 | Invitrogen | 7.5 | 750 | μg/mL |
| Puromycin | A11138-03 | Life | 0.05 | 1 | μg/mL |
| Dihydrochloride | | Technologies | | | |

### Recommended Growth Conditions

Cells were passaged at least twice a week (every 72-96 hours). The final concentration did not surpass 2.0×10⁶ cells/ml, and cells were maintained in a 37° C., 5% CO₂ incubator. Thaw was performed in selection antibiotics-free recovery media without Hygromycin/Puromycin and changed to full growth media after first passage.

### Example 3: Hen Egg Lysozyme Sequence Comparison

Hen Egg Lysozyme (HEL) peptide is a key reagent in the assay. Earlier studies showed that within HEL amino acid residues 48-62, residues D52, 155, Q57, S60 are responsible for the

MHC class II interactions (Dadaglio G, Immunity. 1997 June; 6(6):727-38).

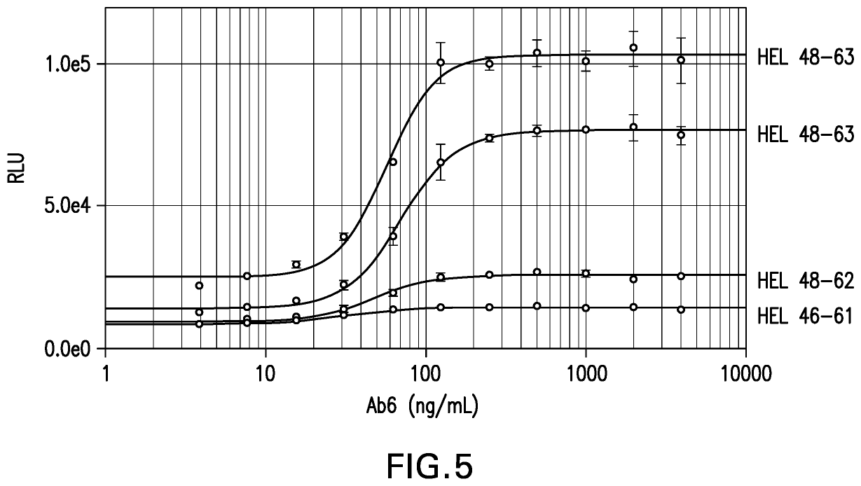

Comparing HEL peptides from different vendors and at various lengths, HEL AA48-63 (DGSTDYGILQINSRWW SEQ ID NO: 20) showed much stronger activation. In contrast, HEL peptides, such as HEL (AA 46-61, NTDGSTDYGILQINSR SEQ ID NO: 21), or HEL (AA 48-62, DGSTDYGILQINSRW SEQ ID NO: 22), did not stimulate the cells to a similar level (See FIG. 5 and Table 3).

TABLE 3
HEL peptide variant tested in the assay system

| | | MW | |
|---|---|---|---|
| Sequence | Purity | (Da) | Stock Concentration |
| HEL 48-63 | ≥98% | 1911 | 0.5 mM in 3% ammonium |
| DGSTDYGILQINSRWW | | | hydroxide |
| (SEQ ID NO: 20) | | | |
| HEL 48-62 | ≥98% | 1726 | 5.79 mM in DMSO |
| DGSTDYGILQINSRW | | | |
| (SEQ ID NO: 22) | | | |
| HEL 46-61 | >95% | 1754 | 0.5 mM in 3% ammonium |
| NTDGSTDYGILQINSR | | | hydroxide |
| (SEQ ID NO: 21) | | | |

### Example 4: Anti-LAG-3 Reporter Gene Functional Assay Protocol Utilizing 3A9-hLAG3-mIL2-Luc2P

### Reagents

HEL AA48-63 (SEQ ID NO: 20 DGSTDYGILQINSRWW) was a custom made peptide from GenScript. The peptide was in a lyophilized powder form in a 1 mg aliquot, and was reconstituted in 3% ammonium hydroxide to make a 500 μM stock. Small aliquots of 20 μl/vial were made and stored at −80° C. until use.

Assay media was constituted of 5% Heat Inactivated FBS (Life Technologies cat #16140-071), 100 U/mL Penicillin-Streptomycin (Life Technologies cat #15140-122) in 94% DMEM with high glucose and HEPES (Life Technologies cat #21063-029).

### Cell Culture and Maintenance

Engineered cell line 3A9-hLAG3-mIL2-Luc2P was cultured in growth medium of Table 2. Engineered cells were maintained in a 37° C. 5% CO₂ incubator and passaged at 3-4 days interval at cell densities not exceeding 2.0×10⁶ cells/mL by the time of splitting. Cell doubling time was calculated based on initial concentration, final concentration, and growth duration. The cell numbers needed for seeding were based on expected culturing time.

TABLE 4

| # of days | Approximate cell seeding density for |
|---|---|
| in culture | routine subculture |
| 2 | 1.2 × 10⁵ cells/mL |
| 3 | 0.3 × 10⁵ cells/mL |
| 4 | 0.1 × 10⁵ cells/mL |

### Assay Protocol

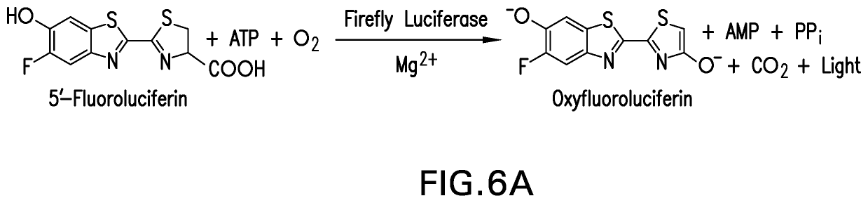

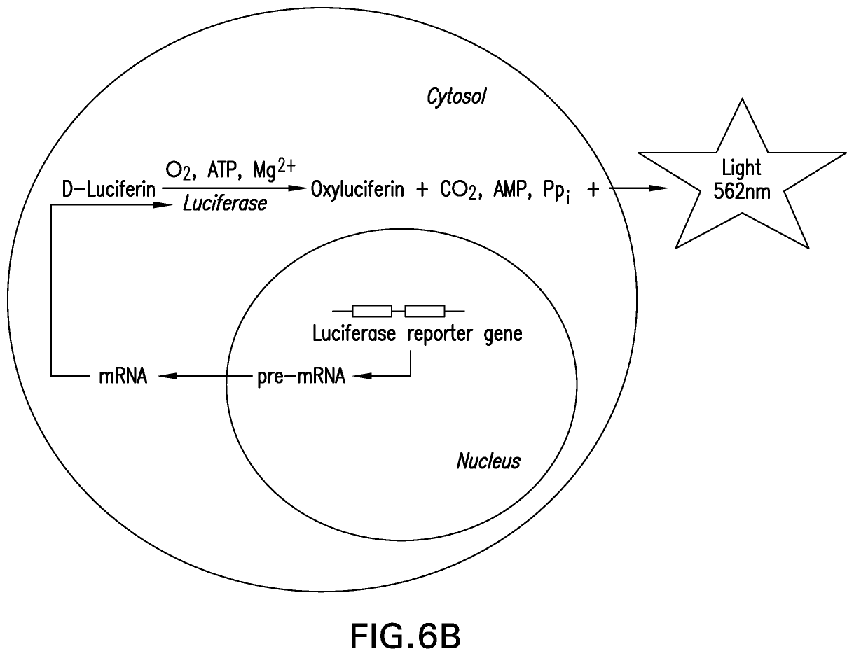

Engineered 3A9-hLAG3-mIL2-Luc2P cells expressing the human LAG3 receptor (CD223) were combined with LK35.2 B cells treated with a HEL peptide and a serial dilution of anti-LAG3 antibody Ab6 reference material or Ab6 samples. After 5 hours incubation, the IL-2 pathway was activated, resulting in IL-2 promoter drived luciferase production. Levels of luciferase were measured in cell lysates using a Promega ONE-Glo™ luciferase Assay System (FIG. 6).

Firefly luciferase is a 61 kDa monomer that catalyzes the mono-oxygenation of beetle luciferin. The enzyme uses ATP as a co-factor although most of the energy for photon production comes from molecular oxygen. The gene encoding firefly luciferase (luc) is a codon optimized cDNA clone that has been incorporated into vectors with cryptic promoters minimized.

### Assay Procedure

Assay media (DMEM High Glucose, HEPES, no phenol red media with 5% FBS HI and 1% Penicillin-Streptomycin solution) was warmed in a water bath set to 37±2° C.

Ab6 reference standards, assay control, and the test sample were diluted to 12 μg/mL in assay media. Serial dilution (1:2.3, 100 μL into 130 μL/well) was performed in a polypropylene dilution plate. 25 μL of prepared Ab6 serial dilutions were transferred to the corresponding wells of the respective white TC treated assay plate. Cells were harvested (spun down and resuspended) and diluted to 3.0×10⁶ cells/mL (3A9-hLAG3-mIL2-Luc2P) and 0.75×10⁶ cells/mL (LK35.2 cell). Equal volumes of 200 nM HEL peptide (diluted from 500 μM in assay media), 3A9-hLAG3-mIL2-Luc2P suspension and LK35.2 cell were combined and 25 μL were added to each well of a 96-well plate. Equal volumes of the diluted suspensions of 3A9-hLAG3-mIL2-Luc2P and LK35.2 cell, and 200 nM HEL peptide solution in a 50 mL were combined in a mixing tube. See Table 5 below scheme based on the number of plates in the assay. Prior to mixing, each of the cell suspension was gently re-suspended by gentle pipetting up and down 3-4 times using a serological pipette. Using a multi-channel pipette, 75 μL of the combined suspension were added to each well of the assay plate(s). The assay plate(s) were placed in a humidified incubator set at 37±1° C. and 5±1% CO₂ for 5 hours ±15 minutes. The assay plates were removed from the incubator and allowed to equilibrate to room temperature for 15 to 20 minutes. Using a multi-channel pipette, 100 μL of ONE-Glo™ substrate solution was added to all wells of the assay plate. The plate(s) were placed on a shaker set to 150-200 rpm for 10-15 minutes at room temperature and covered to protect from light. Luminescence was read on a plate reader SpectraMax M5e or M5 instrument.

TABLE 5

| Number of | Diluted 3A9-hLAG3- | Diluted LK35.2 | 200 nM HEL |
|---|---|---|---|
| plates | mIL2-Luc2P (mL) | cells (mL) | peptide (mL) |
| 1 | 4 | 4 | 4 |
| 2 | 6 | 6 | 6 |
| 3 | 9 | 9 | 9 |
| 4 | 12 | 12 | 12 |

Robustness of the method was evaluated in a design of experiment (DOE) approach to assess impact of method parameters including cell passage number, days in culture, induction incubation time, density of 3A9/LAG-3 cells, density of LK35.2 cells, substrate incubation time, different lots or vendors of key reagents and supplies including Fetal Bovine Serum (FBS), luminescence substrate, HEL peptide, and culture flasks. Data suggest that changes by deliberate variation of tested parameters and different lots or vendors of key reagents have no practical impact on the accuracy of measuring anti-LAG3 antibody potency by the cell-based assay method.

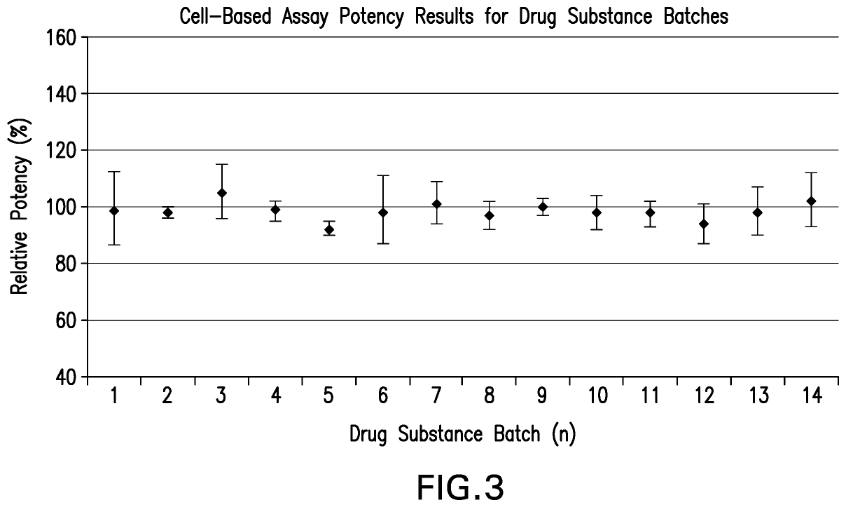

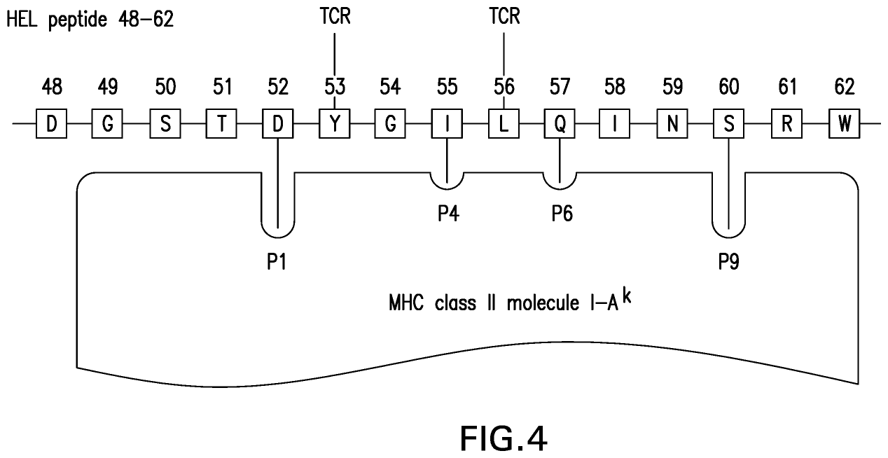

The analytical test method was validated according to ICH Q2 (R1) guidelines. It was demonstrated that the analytical procedure has a remarkable level of accuracy/bias and quantitative precision (<2% and <3% respectively). See FIG. 3. Linearity across the range of the assay was 50% to 200%. The method showed remarkable accuracy/bias and precision (<2% and <3% respectively) for Ab6.

U.S. provisional application 62/951,484 is incorporated herein by reference in its entirety. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A murine T cell 3A9 (ATCC deposit CRL-3293) comprising a polynucleotide sequence encoding human LAG3, a polynucleotide comprising a murine IL-2 promotor sequence operably associated with a reporter gene, wherein upon expression of human LAG3, the human LAG3 is located on the cell surface, wherein the murine IL-2 promotor sequence—lacks the negative regulatory region in −578 to −1219.

2. The murine T cell of claim 1, wherein the murine IL-2 promotor sequence consists of SEQ ID NO: 18 or SEQ ID NO: 19.

3. The murine T cell of claim 1, wherein the polynucleotide sequences are stably transfected.

4. The murine T cell of claim 1, wherein the reporter gene is a firefly luciferase gene.

5. The murine T cell of claim 1, wherein the human LAG3 sequence is SEQ ID NO: 16.

6. A method for measuring the activity of a LAG3 antagonist comprising the steps of:
a) combining suspensions of the murine T cells of claim 1 and LK35.2 B cells (ATCC deposit HB-98) with a Hen Egg Lysozyme (HEL) peptide of SEQ ID NO: 20, 21 or 22, and the LAG3 antagonist in an assay media; and
b) detecting the reporter gene expression through a read-out signal.

7. The method of claim 6, wherein the HEL peptide is SEQ ID NO: 20.

8. The method of claim 6, wherein the LAG3 antagonist is both a reference standard and a test sample, and the method further comprises the step of determining the relative potency of the test sample to the reference standard.

9. The method of claim 6, wherein the reporter gene is a firefly luciferase gene, and 5′-fluoroluciferin is added prior to step b.

10. The method of claim 6, wherein the LAG3 antagonist is an anti-LAG3 antibody or antigen binding fragment.

11. The method of claim 10, wherein the anti-LAG3 antibody or antigen binding fragment comprises: (a) light chain CDRs of SEQ ID NOs: 6, 7 and 8 and (b) heavy chain CDRs of SEQ ID NOs: 9, 10 and 11.

12. The method of claim 11, wherein the anti-LAG3 antibody or antigen binding fragment comprises a heavy chain variable region comprising SEQ ID NO:5 and the light chain comprises a light chain variable region comprising SEQ ID NO: 4.

13. The method of claim 12, wherein the anti-LAG3 antibody comprises a heavy chain and a light chain, and wherein the heavy chain comprises SEQ ID NO:3 and the light chain comprises SEQ ID NO:2.

14. The method of claim 10, wherein the anti-LAG3 antibody is an Ab6 variant.
